(19) United States
(12) Patent Application Publication
(10) Pub. No.: US 2022/0409524 A1
(43) Pub. Date: Dec. 29, 2022

(54) MICRONEEDLE DEVICE

(71) Applicant: HISAMITSU PHARMACEUTICAL CO., INC., Tosu-shi (JP)

(72) Inventors: Ryota HORI, Tsukuba-shi (JP); Toshihiro KOGURE, Tsukuba-shi (JP); Seiji TOKUMOTO, Tsukuba-shi (JP); Shinpei NISHIMURA, Tsukuba-shi (JP)

(21) Appl. No.: 17/902,303

(22) Filed: Sep. 2, 2022

Related U.S. Application Data
(63) Continuation of application No. 16/473,119, filed on Jun. 24, 2019.

(30) Foreign Application Priority Data
Dec. 26, 2016 (JP) 2016-250998

(51) Int. Cl. A61K 9/00; A61K 31/4174; A61K 31/135; A61K 47/20; A61K 47/02; A61M 37/00

(52) U.S. Cl. CPC A61K 9/0021; A61K 31/4174; A61K 31/135; A61K 47/20; A61K 47/02; A61M 37/0015; A61M 2037/0053; A61M 2037/0023

(57) ABSTRACT

A microneedle device of the present invention comprises a substrate, microneedles disposed on the substrate, and a coating formed on the microneedles, wherein the coating comprising dexmedetomidine or a pharmaceutically acceptable salt thereof and isoproterenol or a pharmaceutically acceptable salt thereof. Using said microneedle device, a fast increase rate of dexmedetomidine concentration in plasma after application of the microneedle device is achieved.

### TECHNICAL FIELD

The present invention relates to a microneedle device.

### BACKGROUND ART

Dexmedetomidine acts on α₂ adrenergic receptors so as to exhibit a sedative effect. Dexmedetomidine is used, for example, for sedation during artificial respiration and after removal of the artificial respiration in an intensive care, or for sedation during non-intubated surgery or treatment under local anesthesia. For such applications, dexmedetomidine is provided in a form of hydrochloride as liquid medicine for intravenous injection.

On the other hand, transdermal administration using a microneedle device is known as a form of administering an agent (for example, Patent Literature 1). A microneedle device allows microneedles to make punctures in a stratum corneum layer as the outermost layer of the skin so as to form fine holes through which an agent passes, so that the agent can be transdermally administered. The transdermal administration of dexmedetomidine using a microneedle device has not been conventionally studied.

### CITATION LIST

### Patent Literature

Patent Literature 1: Japanese Unexamined Patent Publication No. 2001-506904

### SUMMARY OF INVENTION

### Technical Problem

The present inventors have attempted administration of dexmedetomidine hydrochloride using a microneedle device to find that the increase rate of dexmedetomidine concentration in plasma is extremely slower than in the case of intravenous injection. A slow increase of dexmedetomidine concentration in plasma makes it difficult to obtain an expected therapeutic effect at an expected time.

### Solution to Problem

Through intensive study, the present inventors have found that blending isoproterenol with dexmedetomidine hydrochloride improves the increase rate of dexmedetomidine concentration in plasma, and have thereby accomplished the present invention.

The present invention is a microneedle device comprising a substrate, microneedles disposed on the substrate, and a coating formed on the microneedles, wherein the coating comprises dexmedetomidine or a pharmaceutically acceptable salt thereof and isoproterenol or a pharmaceutically acceptable salt thereof.

A total amount of isoproterenol and a pharmaceutically acceptable salt thereof may be 0.3 parts by mass or more based on 100 parts by mass of a total amount of dexmedetomidine and a pharmaceutically acceptable salt thereof in the coating.

The coating may further comprise a stabilizer, and the stabilizer may be L-cysteine or sodium pyrosulfite.

### Advantageous Effects of Invention

According to the microneedle device of the present invention, a fast increase rate of dexmedetomidine concentration in plasma after administration of dexmedetomidine or a pharmaceutically acceptable salt thereof is achieved.

### BRIEF DESCRIPTION OF DRAWINGS

FIG. 1 is a perspective view showing a microneedle device in an embodiment.

FIG. 2 is a cross-sectional view taken from line II-II of FIG. 1.

FIG. 3 is a graph showing the change with time in the dexmedetomidine (DEX) concentration in plasma in Test Example 1.

FIG. 4 is a graph showing the change with time in the dexmedetomidine (DEX) concentration in plasma in Test Example 2.

FIG. 5 is a graph showing the change with time in the dexmedetomidine (DEX) concentration in plasma in Test Example 3.

### DESCRIPTION OF EMBODIMENTS

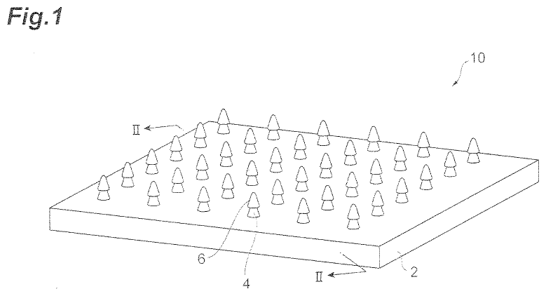

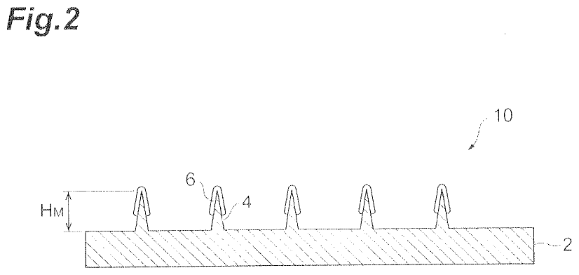

The microneedle device of the present invention comprises a substrate, microneedles disposed on the substrate, and a coating formed on the microneedles. The microneedle device of the present invention in an embodiment is shown in FIG. 1 and FIG. 2. A microneedle device 10 comprises a substrate 2, a plurality of microneedles 4 disposed on the surface of the substrate 2, and a coating 6 formed on the microneedles 4. In the present specification, a structure having microneedles 4 formed on the substrate 2 is referred to as a microneedle array. As the microneedle array, a conventionally known microneedle array may be used. An example of the detail of the microneedle array is as follows.

The substrate 2 is a foundation for supporting the microneedles 4. The shape and the form of the substrate are not particularly limited, and are, for example, a rectangular shape or a circular shape and a flat form or a curved form. The area of the substrate 2 is, for example, 0.5 cm² to 10 cm², or 1 cm² to 3 cm².

The microneedle 4 is a convex structure, which denotes a needle shape in a broad sense or a structure comprising a needle shape. The microneedle 4 is not limited to a structure having a needle shape with a pointed end, and may be in a shape without a pointed end. The microneedle 4 is, for example, in a polygonal pyramid shape such as a quadrangular pyramid shape or a conical shape. The microneedle 4 is a microstructure having a length (height) of, for example, 300 μm to 500 μm.

The microneedles 4 are arranged, for example, in a square lattice form, a rectangular lattice form, an orthorhombic lattice form, a 45-degree staggered form, or a 60-degree staggered form. From the perspective of efficiently introducing dexmedetomidine or a pharmaceutically acceptable salt thereof in the coating 6 into the skin, the number of the microneedles 4 per 1 cm² of a substrate 2 may be 100 to 10000, and the number is preferably 200 to 5000, more preferably 400 to 850.

Examples of the material of the substrate 2 or the microneedle 4 include silicon, silicon dioxide, ceramics, metals, polysaccharides, and synthesized or natural resin materials. More specifically, synthesized or natural resin materials including a biodegradable polymer such as polylactic acid, polyglycolide, polylactic acid-co-polyglycolide, pullulan, caprolactone, polyurethane and polyanhydride, or a non-degradable polymer such as polycarbonate, polymethacrylate, ethylene vinyl acetate, polytetrafluoroethylene and polyoxymethylene are preferred.

The coating 6 may be formed on all of the plurality of microneedles 4 that exist, or may be formed on a part of the microneedles 4 only. The coating 6 may be formed on a tip part only of the microneedle 4, or may be formed to cover the whole of the microneedle 4. The average thickness of the coating 6 may be less than 50 μm, or may be 1 μm to 30 μm.

The coating comprises dexmedetomidine or a pharmaceutically acceptable salt thereof (e.g., hydrochloride) and isoproterenol or a pharmaceutically acceptable salt thereof (e.g., hydrochloride). Hereinafter, “dexmedetomidine or a pharmaceutically acceptable salt thereof” may be referred to as “dexmedetomidine”, and “isoproterenol or a pharmaceutically acceptable salt thereof” may be referred to as “isoproterenol” in some cases. These terms are used as having the same meaning unless a particular distinction is made.

The amount of coating per 1 cm² of a substrate may be 10 μg to 400 μg, or 20 μg to 300 μg. Although depending on the purpose of treatment, the total content of dexmedetomidine and a pharmaceutically acceptable salt thereof in the total amount of coating may be 10 mass % to 90 mass %, and is preferably 40 mass % to 85 mass %, more preferably 60 mass % to 80 mass %, in order to obtain substantial therapeutic effect of dexmedetomidine.

From the perspective of improving the increase rate of dexmedetomidine concentration in plasma, the total amount of isoproterenol and a pharmaceutically acceptable salt thereof based on 100 parts by mass of a total amount of dexmedetomidine and a pharmaceutically acceptable salt thereof in the coating may be 0.3 parts by mass or more, and preferably 1.1 parts by mass or more, more preferably 3.4 parts by mass or more, particularly preferably 6.9 parts by mass or more. The total amount of isoproterenol and a pharmaceutically acceptable salt thereof in the total amount of coating may therefore be, for example, 0.2 mass % or more, 0.8 mass % or more, 2.4 mass % or more, or 4.8 mass % or more. From the perspective of inhibiting side effects of isoproterenol (palpitations and paresthesia of face or scalp), it is preferable that the total amount of isoproterenol and a pharmaceutically acceptable salt thereof in the coating be 1 μg or less. The total amount of isoproterenol and a pharmaceutically acceptable salt thereof may be, for example, 12 mass % or less, 9.5 mass % or less, 9.0 mass % or less, 5.5 mass % or less, 5.0 mass % or less, or 4.8 mass % or less, based on the total amount of coating, and may be 18 parts by mass or less, 8.5 parts by mass or less, 8.0 parts by mass or less, or 7.0 parts by mass or less, based on 100 parts by mass of a total amount of dexmedetomidine and a pharmaceutically acceptable salt thereof.

The coating may further comprise biologically inactive components. The total amount of the biologically inactive components in the total amount of coating is, for example, 0 mass % to 70 mass %. Examples of the biologically inactive components include a base, a stabilizer, a pH adjuster, and other components (e.g., components for accelerating transfer of drugs into blood, a surfactant, oils and fats, or inorganic substances). Also, the coating may further comprise components known as vasodilator such as nicotinic acid amide, in addition to isoproterenol.

The base performs function of retaining a drug to the microneedles, and also increases the viscosity of a coating composition used for forming the coating so as to exhibit an effect of easier application to the microneedle. Examples of the base include water-soluble polymers such as polysaccharides, cellulose derivatives, biodegradable polyester, biodegradable polyamino acid, polyethylene oxide, polyvinyl alcohol, and polyvinylpyrrolidone, and low-molecular weight molecules such as sugar, sugar alcohol and ascorbic acid.

Examples of the stabilizer include L-cysteine, sodium pyrosulfite, sodium hydrogen sulfite, ascorbic acid, ehtylenedimamine tetraacetic acid (EDTA) or salts thereof, and dibutylhydroxytoluene (BHT). Among these, L-cysteine, sodium pyrosulfite and sodium hydrogen sulfite are more preferred from the perspective of stabilizing dexmedetomidine and isoproterenol. These stabilizers may be used alone, or multiple stabilizers may be used in combination.

As the pH adjuster, an inorganic acid or an organic acid, an alkali, a salt, an amino acid or a combination thereof, which is typically used as a pH adjuster for agents, may be used.

Next, a method for producing a microneedle device will be described. A microneedle device is produced by applying a coating composition comprising dexmedetomidine and isoproterenol to microneedles of a microneedle array, and drying the composition to form a coating on the microneedles. Coating may be performed, for example, by filling a reservoir having multiple hollows with the coating composition, and dipping the microneedles therein.

The coating composition may comprise a solvent (water, polyhydric alcohols, lower alcohols, triacetin, etc.) for dissolving dexmedetomidine, isoproterenol and other components that form a coating. All or a part of the solvent is removed in the step of drying the coating composition.

### EXAMPLES

(Preparation of Microneedle Device)

In the following examples, microneedle devices were prepared by the following method.

Each of the components that form a coating and a suitable amount of purified water (and ethanol, if required) were mixed to produce a solution of a coating composition. Subsequently, the tips of the microneedles were dipped in the solution and the solution was air-dried to form a coating on the microneedles. Dipping was performed such that the amount of dexmedetomidine hydrochloride in the coating was controlled at a level of about 30 to 60 μg.

(Analysis of Increase Rate of Dexmedetomidine Concentration)

In the following examples, the increase rate of dexmedetomidine (Dex) concentration in plasma was analyzed as follows.

1) A microneedle device was applied to the skin with hair removed in the ventral region of a male dog, and blood was collected after 5, 10, 20, 30, 60 and 90 minutes.
2) The dexmedetomidine concentration in plasma was obtained by quantifying the dexmedetomidine in plasma by liquid chromatography-mass spectrometry (LC-MS).
3) From the change with time in the value obtained by dividing the dexmedetomidine concentration by the dosage of dexmedetomidine, the increase rate of dexmedetomidine concentration was analyzed. The dosage of dexmedetomidine was calculated from the amount of dexmedetomidine hydrochloride blended in the coating.

(Measurement of Amount of Each Component Transferred into Skin)

In the following examples, each of the components in the coating which had been transferred into the skin was obtained as follows.

1) After a microneedle device was applied for 10 seconds, each of the components remaining on the skin surface and on the microneedle device (e.g., dexmedetomidine hydrochloride or a vasodilator) was collected, and each amount (residual amount) was measured by HPLC method.

2) Separately, another microneedle device having the same features was prepared to measure the amount of each of the components in the coating (initial amount).

3) From the initial amount and the residual amount thus obtained, the amount of each of the components which had been transferred into the skin was calculated.

(Analysis of Storage Stability)

In the following examples, the storage stability of dexmedetomidine hydrochloride or isoproterenol hydrochloride in the coating was determined as follows.

1) A microneedle device after preparation was seal-packed with an aluminum laminate packaging material.

2) After 3 days from preparation of the microneedle device, the amount (initial amount) of dexmedetomidine hydrochloride or isoproterenol hydrochloride in the coating was measured by HPLC method.

3) Separately, a microneedle seal-packed in an aluminum laminate packaging material in the same manner was prepared, and after storage at 50° C. for one week, the amount of dexmedetomidine hydrochloride or isoproterenol hydrochloride in the coating was measured by HPLC method.

### Test Example 1

A microneedle device having a coating comprising components shown in Table 1 was prepared. “Base” in the table is a biologically inactive component comprising a water-soluble polymer (the same shall apply hereinafter). The specification of the microneedle array used is as follows.

Material: polylactic acid

Shape of microneedle: quadrangular pyramid

Length of microneedle: 500 μm

Arrangement of microneedles: lattice form

Number of microneedles: 640

Area of the substrate where microneedles are arranged: about 1 cm²

TABLE 1

| Component | Comparative Example 1 |
|---|---|
| Dexmedetomidine hydrochloride | 68 |
| Base | 18 |
| L-cysteine | 7.5 |
| Total (part by mass) | 93.5 |

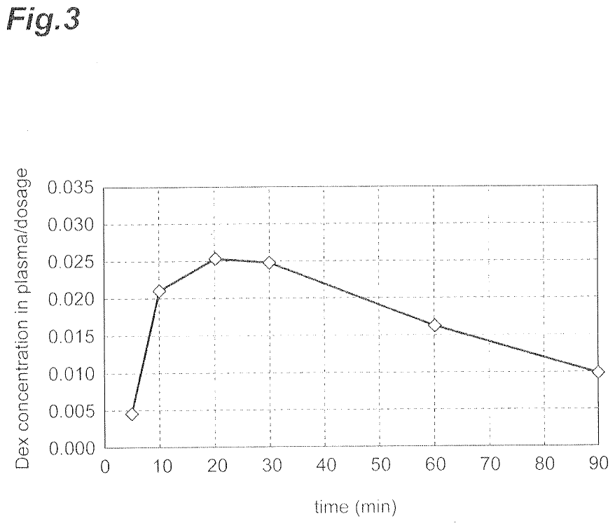

A microneedle was applied to a male dog, and the increase rate of dexmedetomidine concentration in plasma was analyzed by the method described above. The results are shown in FIG. 3. When a microneedle device comprising no isoproterenol hydrochloride was applied, it took time until the dexmedetomidine concentration in plasma increased. The amount of dexmedetomidine hydrochloride transferred into the skin was 58.5 μg in 90 minutes.

### Test Example 2

Microneedle devices having a coating comprising components shown in Table 2 were prepared. The specification of the microneedle array used is as follows.

Material: polylactic acid

Shape of microneedle: quadrangular pyramid

Length of microneedle: 500 μm

Arrangement of microneedles: lattice form

Number of microneedles: 156

Area of the substrate where microneedles are arranged: about 1 cm².

TABLE 2

| | | | Compar- | Compar- | Compar- |
|---|---|---|---|---|---|
| | | Exam- | ative Ex- | ative Ex- | ative Ex- |
| Component | | ple 1 | ample 2 | ample 3 | ample 4 |
| Dexmedetomidine | | 36.5 | 36.6 | 63.1 | 53.6 |
| hydrochloride | | | | | |
| Vasodi- | Isoproterenol | 6.4 | | | |
| lator | hydrochloride | | | | |
| | Terbutaline | | 7.8 | | |
| | sulfate | | | | |
| | Isosorbide | | | 13.4 | |
| | mononitrate | | | | |
| | Nicotinic | | | | 8.5 |
| | acid amide | | | | |

| | | | | |
|---|---|---|---|---|
| Base | 11.5 | 11.5 | 19.7 | 14.6 |
| Total (part by mass) | 54.4 | 55.9 | 96.2 | 76.7 |
| Amount of isoproterenol | 17.5 | — | — | — |
| hydrochloride based on | | | | |
| 100 parts by mass of | | | | |
| dexmedetomidine | | | | |
| hydrochloride (part by | | | | |
| mass) | | | | |

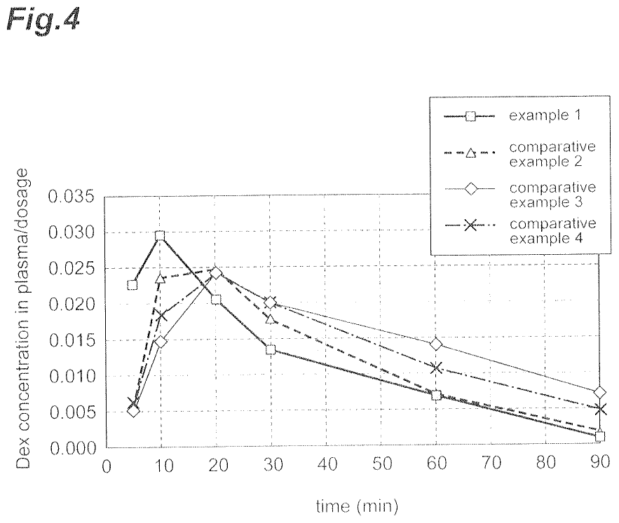

Each microneedle was applied to a male dog, and the increase rate of dexmedetomidine concentration in plasma was analyzed. The results are shown in FIG. 4. When a microneedle device comprising isoproterenol hydrochloride was applied (Example 1), the increase of the dexmedetomidine concentration was faster than in the cases where microneedle devices comprising a vasodilator other than isoproterenol hydrochloride and not comprising isoproterenol hydrochloride were applied (Comparative Examples 2 to 4). The amounts of dexmedetomidine hydrochloride and vasodilators which was transferred into the skin by 10 seconds of application are shown in Table 3.

TABLE 3

| | Amount transferred into skin (μg) | | | |
|---|---|---|---|---|
| | | Compar- | Compar- | Compar- |
| | Exam- | ative Ex- | ative Ex- | ative Ex- |
| Component | ple 1 | ample 2 | ample 3 | ample 4 |
| Dexmedetomidine | 34.1 | 32.8 | 54.1 | 43.9 |
| hydrochloride | | | | |
| Isoproterenol | 6.0 | | | |
| hydrochloride | | | | |
| Terbutaline sulfate | | 7.0 | | |
| Isosorbide mononitrate | | | 11.5 | |
| Nicotinic acid amide | | | | 7.0 |

### Test Example 3

Microneedle devices having a coating comprising components shown in Table 4 were prepared. A microneedle array used was the same as the one in the Test Example 1.

TABLE 4

| | | Exam- | Exam- | Exam- | Exam- |
|---|---|---|---|---|---|
| Component | | ple 2 | ple 3 | ple 4 | ple 5 |
| Dexmedetomidine hydrochloride | | 56.4 | 58.5 | 53.9 | 61.6 |
| Vasodi- | Isoproterenol | 3.9 | 2.0 | 0.6 | 0.2 |
| lator | hydrochloride | | | | |
| | Nicotinic | 10.5 | 10.6 | 9.8 | 11.2 |
| | acid amide | | | | |

| | | | | |
|---|---|---|---|---|
| Base | 11.1 | 11.3 | 11.1 | 12.6 |
| Total (part by mass) | 81.9 | 82.4 | 75.4 | 85.6 |
| Amount of isoproterenol | 6.9 | 3.4 | 1.1 | 0.3 |
| hydrochloride based on 100 parts | | | | |
| by mass of dexmedetomidine | | | | |
| hydrochloride (part by mass) | | | | |

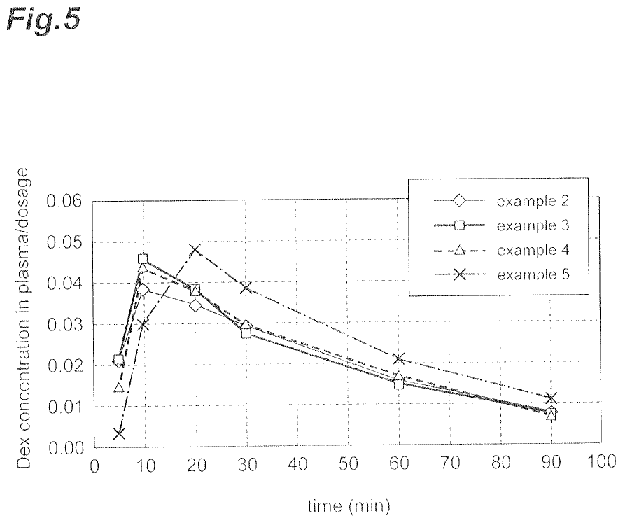

Each microneedle was applied to a male dog, and the increase rate of dexmedetomidine concentration in plasma was analyzed. The results are shown in FIG. 5. When the amount of isoproterenol hydrochloride blended was 0.6 parts by mass or more (Examples 2 to 4), the increase of the dexmedetomidine concentration was faster than in the case where the amount of isoproterenol hydrochloride blended was less than 0.6 parts by mass (Example 5). The amounts of dexmedetomidine hydrochloride and isoproterenol hydrochloride which were transferred into the skin by 10 seconds of application are shown in Table 5.

TABLE 5

| | Amount transferred into skin (μg) | | | |
|---|---|---|---|---|
| | Exam- | Exam- | Exam- | Exam- |
| Component | ple 2 | ple 3 | ple 4 | ple 5 |
| Dexmedetomidine hydrochloride | 49.1 | 52.1 | 46.9 | 51.2 |
| Isoproterenol hydrochloride | 3.0 | 1.8 | 0.5 | 0.17 |

### Test Example 4

Microneedle devices having a coating comprising components shown in Table 6 were prepared. A microneedle array used was the same as the one in the Test Example 1. After storage at 50° C. for one week, the stability of dexmedetomidine hydrochloride in the coating was analyzed by the method described above. Further, the stability of isoproterenol hydrochloride in Examples 7 and 8 was also analyzed.

TABLE 6

| | | | Exam- | Exam- | Exam- |
|---|---|---|---|---|---|
| | | | ple 6 | ple 7 | ple 8 |
| Component | Dexmedetomidine | | 52.6 | 48.1 | 51.8 |
| | hydrochloride | | | | |
| | Vasodi- | Isoproterenol | 4.4 | 4.0 | 4.0 |
| | lator | hydrochloride | | | |
| | | Nicotinic | 8.6 | 7.8 | 8.4 |
| | | acid amide | | | |
| | Base | | 14.7 | 13.4 | 14.5 |
| | Stabi- | Sodium | 1.2 | 0 | 0 |
| | lizer | pyrosulfite | | | |
| | | L-cysteine | 0 | 3.4 | 0 |

| | | | | |
|---|---|---|---|---|
| | Total (part by mass) | 81.5 | 76.7 | 78.7 |
| Result | Amount of dexmedetomidine | 99.7 | 99.4 | 97.9 |
| | hydrochloride after storage | | | |
| | at 50° C. (% based on | | | |
| | initial amount) | | | |
| | Amount of isoproterenol | — | 97.8 | 82.3 |
| | hydrochloride after storage | | | |
| | at 50° C. (% based on | | | |
| | initial amount) | | | |

The results are shown in Table 6. Blending sodium pyrosulfite in the coating resulted in improvement in the stability of dexmedetomidine hydrochloride (Example 6). Blending L-cysteine in the coating resulted in improvement in the stability of dexmedetomidine hydrochloride and isoproterenol hydrochloride (Example 7).

### REFERENCE SIGNS LIST

2: Substrate, 4: Microneedle, 6: Coating, 10: Microneedle device

1-5. (canceled)

6. A method for enhancing plasma concentration of dexmedetomidine in a patient, the method comprising: administering to the patient dexmedetomidine or a pharmaceutically acceptable salt thereof in a microneedle device, the microneedle device comprising:
a substrate;
microneedles disposed on the substrate; and
a coating formed on the microneedles, wherein the coating comprises the dexmedetomidine or the pharmaceutically acceptable salt thereof and isoproterenol or a pharmaceutically acceptable salt thereof,
wherein a total content of the dexmedetomidine or the pharmaceutically acceptable salt thereof in a total amount of the coating is 40 mass % to 90 mass %, and
wherein a total amount of the isoproterenol or the pharmaceutically acceptable salt thereof is 0.3 parts by mass or more based on 100 parts by mass of a total amount of the dexmedetomidine or the pharmaceutically acceptable salt thereof in the coating, effective to enhance a rate of plasma concentration of the dexmedetomidine or the pharmaceutically acceptable salt thereof in the patient.

7. The method for enhancing plasma concentration of dexmedetomidine in a patient according to claim 6, wherein the total amount of the isoproterenol or the pharmaceutically acceptable salt thereof is 1.1 parts by mass or more based on 100 parts by mass of the total amount of the dexmedetomidine or the pharmaceutically acceptable salt thereof in the coating.

8. The method for enhancing plasma concentration of dexmedetomidine in a patient according to claim 6, wherein the coating further comprises a stabilizer.

9. The method for enhancing plasma concentration of dexmedetomidine in a patient according to claim 6, wherein the coating further comprises L-cysteine.

10. The method for enhancing plasma concentration of dexmedetomidine in a patient according to claim 6, wherein the coating further comprises sodium pyrosulfite.
